(19) United States
(12) Patent Application Publication
(10) Pub. No.: US 2018/0299436 A1
(43) Pub. Date: Oct. 18, 2018

(54) A METHOD, KIT AND SYSTEM FOR PREPARING AN ANTIBODY PAIR AND THE USE OF THE KIT

(71) Applicant: SHENZHEN NEW INDUSTRIES BIOMEDICAL ENGINEERING CO., LTD., Shenzhen (CN)

(72) Inventor: Wei RAO, (US)

(21) Appl. No.: 15/540,839

(22) PCT Filed: Jan. 20, 2017
(86) PCT No.: PCT/CN2017/071969

§ 371 Date: Jun. 29, 2017

(51) Int. Cl. G01N 33/541; G01N 33/533; G01N 33/534; G01N 33/535; G01N 33/549; G01N 33/563

(52) U.S. Cl. CPC G01N 33/541; G01N 33/533; G01N 33/563; G01N 33/535; G01N 33/549; G01N 33/534

(57) ABSTRACT

The application discloses a method, kit and system for preparing an antibody pair, and the use of the kit. Wherein the method comprises using an antigen-binding fragment of an existing antibody as a capture antibody and using an anti-crystallizable fragment antibody as a labelled antibody to directly screen a target antibody which can be paired with the existing antibody from cell culture supernatant. By using the technical solution of the present application, the cell culture supernatant can be directly screened to obtain the antibody pair without requiring a large-scale preparation, purification and labelling of the antibody to be screened, thereby greatly reducing the workload.

### TECHNICAL FIELD

The present application relates to the technical field of Biomedicine, specifically, relates to a method, kit and system for preparing an antibody pair and the use of the kit.

### BACKGROUND

The double antibody sandwich method is a technique for quantitative detection of a antigen by using two antibodies binding to the same antigen and is widely used in ELISA, CLIA, RIA and other immunological detection analyses because of its advantages of high sensitivity, strong anti-interference ability, and the like.

In the application process of double antibody sandwich method, on the one hand, the formation of steric hindrance after an antibody binds to an antigen will prevent most of other antibodies binding to the antigen again, and the antibodies that can be successfully paired with each other have stringent requirements on the antibody binding angle and the distance between the identified antigen determinants; on the other hand, each detection system has different requirements on antibody pairs since the solid phases and labels adopted are different, for example, not all antibody pairs that can be used in ELISA can be used in CLIA, which puts forward more requirements on antibody pairs.

Paired antibodies or an antibody pair refers to two identical or different antibodies capable of binding to an antigen simultaneously. At present, the general method of preparing paired antibodies is as the following: 1) preparing a batch of monoclonal antibodies against the target antigen by hybridoma technique, ADLib, or phage antibody library and other methods; 2) obtaining the above antibodies in large quantities by ascites or large-scale cell culture and other approaches; 3) combining all the antibodies in pairs (also their own combinations), one used as a capture antibody immobilized on the ELISA plate, magnetic beads and other solid phase medium, and another one used as a labelled antibody labelled with chromogenic compounds (alkaline phosphatase, horseradish peroxidase or luminol and other substances); 4) detecting the target antigen by the sandwich method, determining whether the pairing is successful according to the signal strength.

This method is also known as the shotgun method in the conventional sense, and has a lot of limiting factors. Firstly, due to manpower limitations, the number of antibodies acquired through step 1) is usually only a few dozens, which greatly limits the range of choices; secondly, the high-sensitivity antibodies obtained from step 1) have a greater possibility of not finding antibody with which they are paired and thus are discarded. Thirdly, the large-scale preparation, purification and labelling of antibodies in the pairing screening process are time-consuming and laborious, and the labelling process may put some influences on the antibodies themselves, thus interfering with judgement.

There are few existing improvements on the method of preparation of paired antibodies. Reference can be made to Chinese patent CN 103884842 A. The improvements comprise: 1) labelling the antibodies to be screened with biotin and chromogenic compounds, respectively, and then coating the avidin and performing pairing detection using sandwich ELISA method; 2) eliminating the interference of the antibody dosage to the detection by the normalization method (i.e., dividing the detection signal by the concentration of the used antibody molecule pair); 3) screening is performed according to the principle that the detection signal of the antibody pair composed of the same antibody molecule is not stronger than the detection signal of the antibody pair composed of a antibody molecule and another antibody molecule. Also, as described in European patent WO 2004025248 A2, paired antibodies are screened by the comparison that the affinity of the complex is greater than the cross-reactivity of the antibodies. All the above methods require the preparation of antibodies in advance, and the effect only relies on the improvement of pairing accuracy.

### SUMMARY OF THE INVENTION

The present application aims to provide a method, kit and system for preparing an antibody pair and the use of the kit, so as to solve the technical problems in the prior art that the preparation of antibodies requires many processes like preparation, purification, labelling and others and the workload is too large.

In order to achieve the above objectives, according to one aspect of the present application, provided is a method of preparing an antibody pair. The method comprises using an antigen-binding fragment of an existing antibody as a capture antibody and using an anti-crystallizable fragment antibody as a labelled antibody to directly screen a target antibody which can be paired with the existing antibody from the cell culture supernatant.

Further, the antigen-binding fragment of the antibody is Fab′ or F(ab)₂, preferably F(ab)₂; and the labelled antibody is an anti-Fc antibody. Further, the method also comprises: optimizing screened antibodies according to a total antibody content in the cell culture supernatant or a binding strength of a antibody to be screened in the cell culture supernatant to a target antigen.

Further, the labelled antibody is labelled with a tracer marker which labels the labelled antibody directly or indirectly; preferably, the tracer marker is selected from at least one of an enzyme label, a fluorescent dye, a chemiluminescent dye, and a radioactive label.

Further, optimizing step comprises: a) detecting the content of the screened antibody, and recording the resulting signal as OD₁; b) detecting the total antibody content in the cell culture supernatant, and recording the resulting signal as OD₂; c) detecting the binding strength of the antibody to be screened in the cell culture supernatant to the target antigen, and recording the resulting signal as OD₃; and d) evaluating and optimizing the antibody screened in the step a) based on the OD₁/OD₂ or OD₁/OD₃ ratio.

Further, the step d) comprises: 1) if the cell culture is a single cell strain cell culture, then the antibody with higher OD₁/OD₂ is paired better with the existing antibody; 2) if the cell culture is multiple cell strains cell culture, then the cell culture with higher OD₁/OD₃ is selected to be separated into single cells for continuous culture so as to obtain new cell cultures, and the new cell cultures is further optimized following the step 1).

Further, the step d) specifically comprises: dividing the cell cultures into two groups according to the single colony wells and the multiple colony wells; sorting the group of the single colony wells according to OD₁/OD₂ from high to low, and selecting the first N cell strains, wherein N is 1-20; sorting the group of the multiple colony wells according to OD₁/OD₃ from high to low, and selecting the first M cell wells for subcloning, respectively, and detecting the subcloned cells according to the methods of the step 1) and the step 3) and selecting N cell strains with higher OD₁/OD₂, wherein M is 1-40.

Further, the step b) specifically comprises: detecting the total antibody content in the cell culture supernatant using an antibody that specifically binds to an antibody conserved region or an immunoglobulin binding protein; preferably, the antibody that specifically binds to an antibody conserved region is selected from the mixture of two or more strains of goat anti-mouse IgM, goat anti-mouse IgA, goat anti-mouse IgD, goat anti-mouse IgG1, goat anti-mouse IgG2a, goat anti-mouse IgG2b, goat anti-mouse IgG3, rabbit anti-mouse IgM, rabbit anti-mouse IgA, rabbit anti-mouse IgD, rabbit anti-mouse IgG1, rabbit anti-mouse IgG2a, rabbit anti-mouse IgG2b and rabbit anti-mouse IgG3, and the immunoglobulin binding protein is a Staphylococcus protein A and/or a Streptococcus G protein.

Further, the step c) specifically comprises: coating the target antigen on a solid phase, and detecting the binding strength of the antibody to be screened in the cell culture supernatant to the target antigen by means of a second antibody.

Further, the existing antibody is any one of the group consisting of an anti-gastrin-17 antibody, an antifolate binding protein antibody, an anti-thyroid peroxidase antibody, an anti-thyroglobulin antibody, an anti-insulin antibody, an anti-ferritin antibody, an serum anti-alpha-fetoprotein antibody, an anti-carcinoembryonic antibody, an anti-prostate specific antigen antibody, an anti-luteinizing hormone antibody, an anti-prolactin antibody, an anti-human chorionic gonadotrnpin antibody, an anti-neuron-specific enolase antibody, an anti-carbohydrate antigen 125 antibody, an anti-carbohydrate antigen 153 antibody, an anti-carbohydrate antigen 199 antibody, an anti-cytokeratin nineteen fragment antibody, an anti-carbohydrate antigen 724 antibody, an anti-carbohydrate antigen 242 antibody, an anti-growth hormone antibody, an anti-myoglobin antibody, an anti-carbohydrate antigen 50 antibody, an anti-C reactive Protein antibody, an anti-corticotropin antibody, an anti-creatine kinase isoenzyme antibody, an anti-Sangtec-100 protein antibody, an anti-laminin antibody, an anti-type IV collagen antibody, an anti-brain natural peptide N-terminal precursor protein antibody, an anti-troponin antibody, an anti-serum parathyroid hormone antibody, an anti-serum calcitonin antibody, an anti-procalcitonin antibody, an anti-prostate acid phosphatase antibody, an anti-osteocalcin antibody, an anti-pregnancy-associated protein A antibody, an anti-pepsinogen I antibody, an anti-pepsinogen II antibody, an anti-insulin-like growth factor antibody or an anti-D-dimer antibody. Further, the method comprises a expansion culture of the cell culture containing the screened antibody and a large-scale preparation and purification of the target antibody.

According to another aspect of the present application, provided is a kit for preparing an antibody pair. The kit comprises: a capture antibody, which is formed by immobilizing the antigen-binding fragment of the existing antibody on a solid phase medium; and a labelled antibody, which is an anti-crystallizable fragment antibody.

Further, the antigen-binding fragment of the antibody is Fab′ or F(ab)₂, preferably F(ab)₂; and the labelled antibody is an anti-Fc antibody. Further, the labelled antibody is labelled with a tracer marker which labels the labelled antibody directly or indirectly; preferably, the tracer marker is selected from at least one of an enzyme label, a fluorescent dye, a chemiluminescent dye, and a radioactive label.

Further, the kit also comprises: an antibody that specifically binds to an antibody conserved region or an immunoglobulin binding protein; preferably, the antibody that specifically binds to an antibody conserved region is selected from the mixture of two or more strains of goat anti-mouse IgM, goat anti-mouse IgA, goat anti-mouse IgD, goat anti-mouse IgG1, goat anti-mouse IgG2a, goat anti-mouse IgG2b, goat anti-mouse IgG3, rabbit anti-mouse IgM, rabbit anti-mouse IgA, rabbit anti-mouse IgD, rabbit anti-mouse IgG1, rabbit anti-mouse IgG2a, rabbit anti-mouse IgG2b and rabbit anti-mouse IgG3, and the immunoglobulin binding protein is a Staphylococcus protein A and/or a Streptococcus G protein.

Further, the kit also comprises: a target antigen which directly or indirectly binds to a solid phase medium.

Further, the solid phase is an ELISA plate, magnetic beads or colloidal gold.

Further, the existing antibody is any one of the group consisting of an anti-gastrin-17 antibody, an antifolate binding protein antibody, an anti-thyroid peroxidase antibody, an anti-thyroglobulin antibody, an anti-insulin antibody, an anti-ferritin antibody, an anti-alpha-fetoprotein antibody, an anti-carcinoembryonic antibody, an anti-prostate specific antigen antibody, an anti-luteinizing hormone antibody, an anti-prolactin antibody, an anti-human chorionic gonadotrnpin antibody, an anti-neuron-specific enolase antibody, an anti-carbohydrate antigen 125 antibody, an anti-carbohydrate antigen 153 antibody, an anti-carbohydrate antigen 199 antibody, an anti-cytokeratin nineteen fragment antibody, an anti-carbohydrate antigen 724 antibody, an anti-carbohydrate antigen 242 antibody, an anti-growth hormone antibody, an anti-myoglobin antibody, an anti-carbohydrate antigen 50 antibody, an anti-C reactive Protein antibody, an anti-corticotropin antibody, an anti-creatine kinase isoenzyme antibody, an anti-Sangtec-100 protein antibody, an anti-laminin antibody, an anti-type IV collagen antibody, an anti-brain natural peptide N-terminal precursor protein antibody, an anti-troponin antibody, an anti-parathyroid hormone antibody, an anti-calcitonin antibody, an anti-procalcitonin antibody, an anti-prostate acid phosphatase antibody, an anti-osteocalcin antibody, an anti-pregnancy-associated protein A antibody, an anti-pepsinogen I antibody, an anti-pepsinogen II antibody, an anti-insulin-like growth factor antibody or an anti-D-dimer antibody. Further, the kit also comprises: reagents used in a expansion culture of cell cultures, and a large scale preparation and purification of the target antibody.

According to a further aspect of the present application, provided is the above described kit used in the preparation of an antibody pair.

Further, the kit is applied to a semi-automatic or full-automatic immunoassay analyzer.

According to yet another aspect of the present application, provided is a system for preparing an antibody pair. The system comprises any one of the above described kits and a semi-automatic or full-automated immunoassay analyzer.

Applying the technical solution of the present application, using an antigen-binding fragment of an existing antibody as a capture antibody and using an anti-crystallizable fragment antibody as a labelled antibody to directly screen a target antibody which can be paired with said existing antibody from cell culture supernatant, without requiring the large-scale preparation, purification and labelling of the antibodies to be screened, thereby greatly reducing the workload.

### BRIEF DESCRIPTION OF THE DRAWINGS

The accompany drawings of the description constituting part of the present application are used to provide a further understanding of the present application; and exemplary embodiments and illustrations thereof of the present application are used to explain the present application, and do not unduly limit the present application. In the drawings:

FIG. 1 shows a schematic diagram of screening an antibody that can be paired with a capture antibody from the cell culture supernatant by sandwich method according to an embodiment of the present application;

FIG. 2 shows a schematic diagram of quantitatively detecting the total antibody content in the cell culture supernatant using a double antibody sandwich method according to an embodiment of the present application; and

FIG. 3 shows a schematic diagram of detecting the binding strength of the antibodies to be screened in the cell culture supernatant to the target antigen using an indirect assay according to an embodiment of the present application.

### DETAILED DESCRIPTION OF THE EMBODIMENTS

It should be noted that, in the case of no conflict, the embodiments of the present application and the features of the embodiments may be combined with each other. The present application will be described in detail below with reference to the accompanying drawings and in combination with the embodiments.

It is well known that in the process of preparing a monoclonal antibody by using the hybridoma method, thousands to millions of cell colonies can be obtained in one cell fusion. The inventors of the present application have found that, in the existing method for preparing paired antibodies, the antibodies available for selection are limited to the antibodies which have been prepared and the large-scale preparation, purification and labelling of all antibodies to be screened are required. In response, the inventors have proposed that if paired antibodies can be screened directly from the cell culture supernatant, the success rate will be increased significantly.

In view of the above described concept and technical problems, the present application aims to provide a method for performing pairing screening directly from the cell culture supernatant and establishing a reliable, real-time evaluation system for optimization as the number of antibodies available for screening increases.

Abbreviations and terms used in the present application are explained below:

Solid phase carrier: it refers to a medium used to immobilize antibodies or antigen in the detection system, such as ELISA plate, magnetic beads, colloidal gold, cellulose acetate film and the like.

Fab: Fab is the antigen-binding fragment of an antibody, equivalent to two arms of a Y-shaped structure, and comprises the intact variable domain and the CH1 domain. Fab′ contains only one arm and is monovalent, and F(ab)₂ contains the entire two arms and is bivalent.

Fc: Fc is a crystallizable fragment, equivalent to the CH2 and CH3 domains of the antibody conserved region.

ELISA: Enzyme Linked Immunosorbent Assay (ELISA) refers to a qualitative and quantitative detection method in which a soluble antigen or antibody is bond to a solid phase carrier such as polystyrene, and an immune reaction is carried out utilizing the antigen-antibody binding specificity.

CLIA: Chemiluminescence Immunoassay (CLIA) is a technology that combines highly sensitive chemiluminescence assay techniques with highly specific immune reactions for the detection and analysis of a variety of antigens, haptens, antibodies, hormones, enzymes, fatty acids, vitamins, drugs and others.

RIA: Radioimmunoassay (RIA) is an analytic method of using radioisotope as a tracer which is labelled on the antigen and using a specific antibody as a conjugate.

ADLib: Autonomously diversifying library (ADLib) is an antibody preparation technique based on a high-frequency transformation of artificially induced antibody genes to form an antibody library.

SPA: Staphylococcus protein A can bind to the Fc fragments of human IgGs and a variety of mammalian IgGs.

SPG: Protein G is a cell wall protein isolated from G type Streptococcus. It can bind to the Fc region of a variety of mammalian IgGs, but can not bind to IgA and IgM.

Tris: trihydroxymethyl aminomethane, weakly basic, can be added to HCl to adjust pH.

RPMI-1640: RPMI is the abbreviation for the Roswell Park Memorial Institute, and refers to a type of cell culture medium developed by this Institute, and 1640 is the code of the culture medium.

CBS: Carbonate buffer solution (CBS) is formulated with appropriate proportions of sodium carbonate and sodium bicarbonate.

Paired antibodies or an antibody pair: it refers to two identical or different antibodies capable of binding to an antigen simultaneously.

Capture antibody: it refers to an antibody that is immobilized to a ELISA plate, magnetic beads, colloidal gold and other solid phase mediums, and can capture the antigen in the capture solution.

Labelled antibodies: The binding properties of the antibody are detected by virtue of labelling the chromogenic compounds such as alkaline phosphatase, horseradish peroxidase, luminol and the like on the antibody.

The second antibody, that is the so-called secondary antibody, can bind specifically to the primary antibody. When the primary antibody is not conducive to labelling in a complex environment, such as the cell culture supernatant, a secondary antibody may be incorporated to label the chromogenic compounds for detection.

Sandwich method: a method of combined detection of an antigen using an antibody pair, or a method of combined detection of an antibody using an antibody pair.

Indirect method: a method of immobilizing antigen to the solid phase carrier, and being detected with the second antibody.

Subcloning: Cells were dispersed by limiting dilution method into individual cells for culture, thereby purifying the cell strain.

Phage antibody library technology: an antibody preparation technology of taking the full set of genes of antibody variable region by PCR amplification, and expressing and screening the target antibody by means of phage surface display technology.

Screening paired antibodies can only be detected based on the sandwich method. If there are an antibody A and an antigen X to which the antibody A specifically bind, it need screen another antibody B paired with A and binding to the antigen X; then A and B act as a capture antibody and a labelled antibody, respectively. If the sandwich detection of the antigen is possible, the pairing is successful. The present application contemplates to directly screen an antibody paired with A from the cell culture supernatant, i.e., the target antibody B is potentially present in the cell culture supernatant. However, there are the following two problems: \hat{1} since the supernatant contains a large amount of impure proteins, the efficiency of pairing will be extremely low, regardless of the cell culture is used as a capture antibody or a labelled antibody; \hat{2} A is used as a capture antibody, and then the antibody B-containing cell culture supernatant is added after capturing the antigen, and then the labelled antibody is incorporated to bind to the antibody B, and due to the high degree of homology between antibody A and antibody B (unless the two antibodies are selected from different species, but this is rather rare), the labelled antibody may also bind to antibody A and thus cause false positive.

The inventive idea of the present application is as follows: using the antigen-binding fragment of antibody A as a capture antibody, adding antibody-B-containing cell culture supernatant after capturing antigen X, and then using an anti-crystallizable fragment as a labelled antibody. The labelled antibody binds only to the Fc fragment of the antibody, so it binds only to B to avoid false positive. Therefore, using an antigen-binding fragment of an existing antibody (such as Fab fragment) as a capture antibody and using an anti-crystallizable fragment (such as Fc fragment) antibody as a labelled antibody to directly screen the antibody that can be paired with the existing antibody from the cell culture supernatant. Then, evaluating and optimizing the screened antibody in the above step according to the total antibody content in the cell culture supernatant or the binding strength of the antibody to be screened in the cell culture supernatant to the target antigen.

According to a typical embodiment of the present application, provided is a method of preparing an antibody pair. The method comprises using an antigen-binding fragment of an existing antibody as a capture antibody and using an anti-crystallizable fragment antibody as a labelled antibody to directly screen a target antibody which can be paired with the existing antibody from the cell culture supernatant.

Wherein, “an existing antibody” refers to a prepared antibody to be screened for pairing with it, but not specifically refer to antibodies in the prior art.

Applying the technical solution of the present application, the cell culture supernatant can be directly screened to obtain the target antibody which can be paired with the existing antibody without requiring the large-scale preparation, purification and labelling of the antibodies to be screened using an antigen-binding fragment of the existing antibody as a capture antibody and using an anti-crystallizable fragment antibody as a labelled antibody, thereby greatly reducing the workload.

The sandwich method used for screening antibodies in the present application may be an enzyme-linked immunosorbent sandwich assay, a chemiluminescent immunoassay sandwich method, or other sandwich detection methods.

According to a typical embodiment of the present application, the antigen-binding fragment is a Fab fragment; the labelled antibody is an anti-Fc antibody. Of course, in the present application, the antibody is not limited to an IgG antibody, but may be other types of antibody. Accordingly, the capture antibody is an antigen-binding fragment thereof and the labelled antibody is an anti-crystallizable antibody. The capture antibody includes, but not limited to, Fab′ or F(ab)₂, and more preferably F(ab)₂, because F(ab)₂ is more stable than Fab′. The capture antibody can be obtained by digesting intact antibodies with pepsin or papain, or can be obtained by genetic engineering method.

According to a typical embodiment of the present application, the method also comprises: optimizing the screened antibody according to the total antibody content in the cell culture supernatant or a binding strength of an antibody to be screened in the cell culture supernatant to a target antigen, so as to obtain an antibody that paired well with the existing antibody.

According to a typical embodiment of the present application, the labelled antibody is labelled with a tracer marker which labels the labelled antibody directly or indirectly; preferably, the tracer marker includes but not limited to at least one of an enzyme label, a fluorescent dye, a chemiluminescent dye, and a radioactive label.

Preferably, preferred steps comprise: a) detecting the content of the screened antibody, and recording the resulting signal as OD₁; b) detecting the total antibody content in the cell culture supernatant, and recording the resulting signal as OD₂; c) detecting the binding strength of the antibody to be screened in the cell culture supernatant to the target antigen, and recording the resulting signal as OD₃; and d) evaluating and optimizing the antibody screened in the step a) based on the OD₁/OD₂ or OD₁/OD₃ ratio.

Wherein, in step a), the content of the screened antibody is detected, and the detected signal is recorded as OD₁. The positive signal indicates that the cell culture to be detected contains the antibody that can be paired with the existing antibody.

In step b), since the amount of effective antibody in the cell culture supernatant is not equal, the strong signal of the culture medium detected in the step a) may be resulted from the large amount of antibody rather than better pairing effect. Therefore, the present application contemplates to obtain the relative value of the antibody amount in the cell culture supernatant, and believe that the antibody with lower effective total antibody content but higher OD₁ has better pairing effect. In this step, the total antibody content in the cell culture (cell culture solution) supernatant is detected, and the resulting signal is recorded as OD₂.

In the step c), the binding strength of the antibody to be screened in the cell culture supernatant to the target antigen is detected.

In the step d), the antibody screened in the step a) is evaluated or optimized based on the OD₁/OD₂ or OD₁/OD₃ ratio.

According to a typical embodiment of the present application, the step d) comprises: 1) if the cell culture is a single cell strain cell culture, then the antibody with higher OD₁/OD₂ is paired better with the existing antibody; 2) if the cell culture is a multiple cell strains cell culture, then the cell culture with higher OD₁/OD₃ is selected to be separated into single cells for continuous culture so as to obtain new cell cultures, and the new cell cultures is further optimized following the step 1). The higher OD₁/OD₃ is, the signal OD₁ that is detected for the screened antibody is closer to the signal OD₃ that is detected for the binding strength of this antibody to the target antigen, indicating that these two antibodies have less interference from each other and better pairing effect.

Preferably, the step d) specifically comprises: dividing the cell cultures into two groups according to the single colony wells and the multiple colony wells; the group of the single colony wells is sorted according to OD₁/OD₂ from high to low, and the first N cell strains are selected, wherein N is 1-20, preferably 10; the group of the multiple colony wells is sorted according to OD₁/OD₃ from high to low, and the first M cell wells are selected respectively for subcloning, and the subcloned cells are detected according to the methods of the step 1) and the step 3) and N cell strains with higher OD₁/OD₂ are selected, wherein M is 1-40, preferably 20.

According to a typical embodiment of the present application, the step b) specifically comprises: detecting the total antibody content in the cell culture supernatant using an antibody that specifically binds to an antibody conserved region or an immunoglobulin binding protein; preferably, the antibody that specifically binds to an antibody conserved region includes but not limited to the mixture of two or more strains of goat anti-mouse IgM, goat anti-mouse IgA, goat anti-mouse IgD, goat anti-mouse IgG1, goat anti-mouse IgG2a, goat anti-mouse IgG2b, goat anti-mouse IgG3, rabbit anti-mouse IgM, rabbit anti-mouse IgA, rabbit anti-mouse IgD, rabbit anti-mouse IgG1, rabbit anti-mouse IgG2a, rabbit anti-mouse IgG2b and rabbit anti-mouse IgG3, and the immunoglobulin binding protein includes but not limited to a Staphylococcus protein A and/or a Streptococcus G protein. The antibody specifically binding to the antibody conserved region that used in this step is selected from the mixture of two or more strains of the above described antibodies.

According to a typical embodiment of the present application, the step c) utilizes indirect method to detect the binding of only the antibody in the culture and the antigen, and specifically comprises: coating the target antigen on a solid phase, and detecting the binding strength of the antibody in the cell culture supernatant to the target antigen by means of a second antibody. In the detection of the indirect method, only the antibody in the culture binds to the antigen, which is different from the effect of the steric hindrance of another antibody in the sandwich method. Therefore, the signal intensity detected in the indirect method is theoretically higher than that in the sandwich method. When the two values are equivalent, these two antibodies do not affect each other for binding to the antigen, and the pairing effect is the best (in the actual operation, due to the environmental difference of the detection, it is desirable to compare the ratio of value obtained in the sandwich method to that obtained in the indirect method).

According to a typical embodiment of the present application, the existing antibody is any one of the group consisting of an anti-gastrin-17 antibody, an antifolate binding protein antibody, an anti-thyroid peroxidase antibody, an anti-thyroglobulin antibody, an anti-insulin antibody, an anti-ferritin antibody, an anti-alpha-fetoprotein antibody, an anti-carcinoembryonic antibody, an anti-prostate specific antigen antibody, an anti-luteinizing hormone antibody, an anti-prolactin antibody, an anti-human chorionic gonadotrnpin antibody, an anti-neuron-specific enolase antibody, an anti-carbohydrate antigen 125 antibody, an anti-carbohydrate antigen 153 antibody, an anti-carbohydrate antigen 199 antibody, an anti-cytokeratin nineteen fragment antibody, an anti-carbohydrate antigen 724 antibody, an anti-carbohydrate antigen 242 antibody, an anti-growth hormone antibody, an anti-myoglobin antibody, an anti-carbohydrate antigen 50 antibody, an anti-C reactive Protein antibody, an anti-corticotropin antibody, an anti-creatine kinase isoenzyme antibody, an anti-Sangtec-100 protein antibody, an anti-laminin antibody, an anti-type IV collagen antibody, an anti-brain natural peptide N-terminal precursor protein antibody, an anti-troponin antibody, an anti-parathyroid hormone antibody, an anti-calcitonin antibody, an anti-procalcitonin antibody, an anti-prostate acid phosphatase antibody, an anti-osteocalcin antibody, an anti-pregnancy-associated protein A antibody, an anti-pepsinogen I antibody, an anti-pepsinogen II antibody, an anti-insulin-like growth factor antibody or an anti-D-dimer antibody.

According to a typical embodiment of the present application, the method also comprises an expansion culture of the cell culture containing the screened antibody and a large-scale preparation and purification of the target antibody.

According to a typical embodiment of the present application, provided is a kit of preparing an antibody pair. The kit comprises a capture antibody formed by immobilizing the antigen-binding fragment of an existing antibody on a solid phase medium and an anti-crystallizable fragment antibody used as a labelled antibody, which are used to directly screen a target antibody which can be paired with the existing antibody from the cell culture supernatant.

According to a typical embodiment of the present application, the antigen-binding fragment is a Fab fragment; the labelled antibody is an anti-Fc antibody. Of course, in the present application, the antibody is not limited to an Ig antibody, but can be other types of antibody. Accordingly, the capture antibody is the antigen-binding fragment thereof and the labelled antibody is the anti-crystallizable antibody thereof. Preferably, the capture antibody is Fab′ or F(ab)₂, and more preferably F(ab)₂, because F(ab)₂ is more stable than Fab′. The capture antibody can be obtained by digesting intact antibodies with pepsin or papain, or can be obtained by genetic engineering method.

According to a typical embodiment of the present application, the labelled antibody is labelled with a tracer marker which labels the labelled antibody directly or indirectly; preferably, the tracer marker is selected from at least one of an enzyme label, a fluorescent dye, a chemiluminescent dye, and a radioactive label.

According to a typical embodiment of the present application, the kit also comprises: an antibody that specifically binds to an antibody conserved region or an immunoglobulin binding protein, used for detecting the total antibody content in the cell culture supernatant; preferably, the antibody that specifically binds to an antibody conserved region includes but not limited to the mixture of two or more strains of goat anti-mouse IgM, goat anti-mouse IgA, goat anti-mouse IgD, goat anti-mouse IgG1, goat anti-mouse IgG2a, goat anti-mouse IgG2b, goat anti-mouse IgG3, rabbit anti-mouse IgM, rabbit anti-mouse IgA, rabbit anti-mouse IgD, rabbit anti-mouse IgG1, rabbit anti-mouse IgG2a, rabbit anti-mouse IgG2b and rabbit anti-mouse IgG3, and the immunoglobulin binding protein includes but not limited to a Staphylococcus protein A and/or a Streptococcus G protein.

According to a typical embodiment of the present application, the kit also comprises: a target antigen that directly or indirectly binds to the solid phase medium, for detecting the binding strength of only the antibody in the culture with the antigen by means of a second antibody using the indirect method.

According to a typical embodiment of the present application, the solid phase is an ELISA plate, magnetic beads or colloidal gold.

According to a typical embodiment of the present application, the existing antibody is any one of the group consisting of an anti-gastrin-17 antibody, an anti-folate binding protein antibody, an anti-thyroid peroxidase antibody, an anti-thyroglobulin antibody, an anti-insulin antibody, an anti-ferritin antibody, an serum anti-alpha-fetoprotein antibody, an anti-carcinoembryonic antibody, an anti-prostate specific antigen antibody, an anti-luteinizing hormone antibody, an anti-prolactin antibody, an anti-human chorionic gonadotrnpin antibody, an anti-neuron-specific enolase antibody, an anti-carbohydrate antigen 125 antibody, an anti-carbohydrate antigen 153 antibody, an anti-carbohydrate antigen 199 antibody, an anti-cytokeratin nineteen fragment antibody, an anti-carbohydrate antigen 724 antibody, an anti-carbohydrate antigen 242 antibody, an anti-growth hormone antibody, an anti-myoglobin antibody, an anti-carbohydrate antigen 50 antibody, an anti-C reactive Protein antibody, an anti-corticotropin antibody, an anti-creatine kinase isoenzyme antibody, an anti-Sangtec-100 protein antibody, an anti-laminin antibody, an anti-type IV collagen antibody, an anti-brain natural peptide N-terminal precursor protein antibody, an anti-troponin antibody, an anti-serum parathyroid hormone antibody, an anti-serum calcitonin antibody, an anti-procalcitonin antibody, an anti-prostate acid phosphatase antibody, an anti-osteocalcin antibody, an anti-pregnancy-associated protein A antibody, an anti-pepsinogen I antibody, an anti-pepsinogen II antibody, an anti-insulin-like growth factor antibody or an anti-D-dimer antibody.

According to a typical embodiment of the present application, the kit also comprises: the reagents used in an expansion culture of cell cultures and a large-scale preparation and purification of the target antibody.

According to a typical embodiment of the present application, provided is a kit for use in preparing an antibody pair.

According to a typical embodiment of the present application, the kit is applied to a semi-automatic or full-automatic immunoassay analyzer.

Specifically, in a typical embodiment of the present application, a method of efficiently preparing an antibody pair using a Fab fragment of a high-sensitive antibody as a capture antibody and an anti-Fc antibody as a labelled antibody is as the following steps:

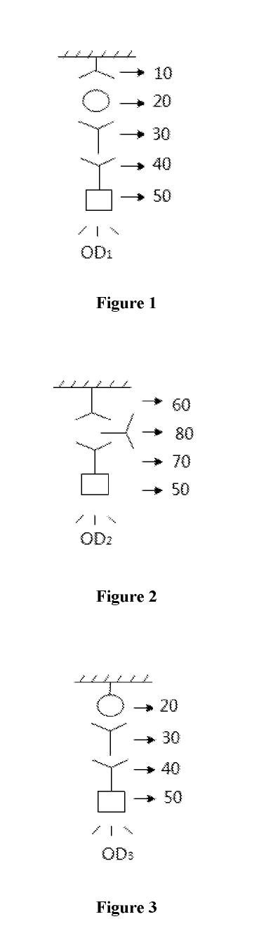

a) As shown in FIG. 1, using the Fab fragment of the existing high-sensitivity antibody as a capture antibody 10, and using the anti-Fc antibody as a labelled antibody 40 (carrying a label 50), from the cell culture supernatant directly screen the antibody 30 (20 is the antigen) that can be paired with the capture antibody by the sandwich method, and the signal detected by the sandwich method is recorded as OD₁;

b) As shown in FIG. 2, the total antibody content in the cell culture supernatant is quantitatively detected using a double antibody sandwich method, that is the total antibody 80 content in the cell culture supernatant is detected by the sandwich method using two antibodies that specifically bind to the antibody conserved region (the second antibody A 60 and the second antibody B 70) and the resulting signal is recorded as OD₂;

c) As shown in FIG. 3, the binding strength of the antibody 30 in the cell culture supernatant to the target antigen 20 is detected by the indirect method (also by the double antigen sandwich method), that is, the target antigen is coated on the solid phase, the binding strength of the antibody in the culture to the antigen is detected by means of the labelled antibody 40 (carrying a label 50), and the resulting signal is recorded as OD₃.

d) the antibody screened in the step a) is evaluated or optimized based on the OD₁/OD₂ or OD₁/OD₃ ratio.

In the step d), if the cell culture is a single cell strain cell culture, then the antibody with higher OD₁/OD₂ is paired better with the existing antibody; if the cell culture is a multiple cell strains cell culture, then the cell culture with higher OD₁/OD₃ is selected to be separated into single cells for continuous culture so as to obtain new cell cultures, and the new cell cultures is further optimized following the step 1). The higher OD₁/OD₃ is, the results of the sandwich method are closer to that of the indirect method, indicating that these two antibodies have less interference from each other and better pairing effect.

The beneficial effect of the present application will be further explained with reference to the examples below.

Pepsin was from Sigma company;

Goat anti-mouse IgG Fc was from Beijing Biodragon Immunotechnologies Co., Ltd. in China;

The G17 antigen was synthesized by the Shanghai Science Peptide Biological Technology Co., Ltd. in China.

The TPO antigen and FABP antigen was expressed and obtained by the Shenzhen New Industrial Biomedical Engineering Co., Ltd. (Snibe) in China;

TMB substrate was from Beijing Biodragon Immunotechnologies Co., Ltd.;

ABEI (N-(4-aminobutyl)-N-ethylisoluminol) was from the Shenzhen New Industrial Biomedical Engineering Co., Ltd. (Snibe) in China;

Magnetic micro-beads was from the Shenzhen New Industrial Biomedical Engineering Co., Ltd. (Snibe) in China.

### EXAMPLE 1

The Comparison of the Effect of Fab′ and F(ab)₂ Coating ELISA Plates

1) Acquisition of Fab′ and F(ab)₂

The antibody Fab′ was acquired by digesting the intact antibody with papain and F(ab)₂ was acquired by digesting the intact antibody with pepsin, respectively, with reference to the method provided by Short Protocols in Immunology (John E. Coligan, USA, 2009). The sandwich assays were performed using anti-thyroid peroxidase (TPO) antibody pair present in the laboratory, which were T-1 and T-2.

Specifically, 2 mL of 2 mg/mL T-1 dissolved in PBS (pH 7.4) was added to 2 mL of 0.1 mg/mL papain dissolved in a hydrolysis buffer (0.02 mol/L EDTA·Na₂, 0.02 mol/L cysteine, PBS), and incubated in bath at 37° C. for 8 h. The iodoacetamide was added to a final concentration of 0.03 mol/L to terminate the hydrolysis reaction. The reaction solution was transferred to a dialysis bag and dialyzed against 2 L PBS (pH 8.0) at 4° C. for 12 h. The dialyzed liquid was loaded using a 5 mm×100 mm protein A cross-linked agarose gel CL-4B chromatographic column, and a liquid flow containing Fab fragments and enzymes was collected and concentrated to 5 mL. The concentrated solution was loaded onto a 26 mm×900 mm polyacrylamide dextran S-200 Superfine chromatographic column and the fraction with the molecular weight of 50 kDa, which was determined by SDS-PAGE, was collected. 20 μL final products was used to determine the purity of the final products by 10% non-reducing SDS-polyacrylamide gel electrophoresis, and the A₂₈₀ value was detected to determine the concentration of Fab′ fragment.

2 mL of 3 mg/mL T-1 was dialyzed against 200 mL of acetic acid buffer (pH 4.0) at 4° C. for 4 h, the concentration was re-measured and adjusted to 2 mg/mL using acetate buffer with the same pH. The pepsin concentration of 0.1 mg/mL was formulated with the acetate buffer (pH 4.0), and 1 mL of the solution was added to the above described antibody solution to make the enzyme/antibody of 1:20 to react at 37° C. for 4 h. The reaction was terminated by adding 100 μL of 2 mol/L Tris base. The mixture was transferred to a dialysis bag and dialyzed against 1 L PBS of pH 8.0 at 4° C. The dialysate was loaded onto a 5 mm×100 mm protein A cross-linked agarose gel CL-4B chromatographic column and the unbound effluent is collected. The effluent was concentrated to 4 mL and loaded onto a 26 mm×900 mm polyacrylamide dextran S-200 Superfine chromatographic column to collect the component with the molecular weight of 110 kDa. The above described product was identified using 10% SDS non-reducing polyacrylamide gel electrophoresis, showing a band only at 110 kDa, a single band at 25 kDa was shown using non-reducing polyacrylamide gel electrophoresis. A₂₈₀ was detected to determine the final concentration of F(ab)₂.

2) T-2 Antibody Labelled with HRP

Reference is made to the method provided by Short Protocols in Immunology (John E. Coligan, USA, 2009). Specifically, 2 mg HRP weighted was dissolved in 0.5 ml water, 0.2 ml of freshly prepared 0.06 mol/L NaIO₄ was added, and then 0.2 mL of 0.16 mol/L ethylene glycol was added, mixed, and allowed to stand at room temperature for 30 min. 0.5 mg T-2 was dialyzed against 0.1 mol/L CBS of pH 9.5 at 4° C. for 2 h, 225 μL HRP solution oxidized by NaIO₄ in the last step was immediately added, stirred at room temperature in dark for 2 h. 40 μL of freshly prepared 2 mg/mL NaBH₄ was added, mixed and standed at 4° C. for 2 h, and the above described solution was loaded into a dialysis bag and dialyzed against 0.01 mol/L pH 7.4 PBS overnight.

3) The Pairing Experiment of Fab′ and F(ab)₂ After Coating the ELISA Plates with T-2

Using CBS pH 9.6, the Fab′, F(ab)₂ and T-1 intact antibodies were diluted to 1 μg/mL, 1 μg/mL and 1.36 mg/mL, respectively, so that the molar concentration is uniform. Added them to each plate at 100 μL/well, 3 wells for each antibody, incubated overnight at 4° C., and washed three times with PBST (containing 0.05% Tween-20 in PBS buffer, pH 7.4). Added 200 μL of 1% OVA (ovalbumin) to incubate at 37° C. for 2 h to block the un-reacted site on the ELISA plate and washed once with PBST. 100 μL of 0.01 μg/mL, 0.1 μg/mL and 1 μg/mL TPO antigens were added to each group, respectively, incubated at 37° C. for 1 h, and washed three times with PBST. 100 μL of 0.1 μg/mL HRP labelled T-2 antibody was added, incubated at 37° C. for 1 h, and washed three times with PBST. Added 100 μL TMB substrate and incubated at room temperature for 10 min, and 50 μL of 2 M H₂SO₄ was added to terminate the reaction. The absorbance at a wavelength of 450 nm was measured.

4) The Pairing Experiment of Fab′ and F(ab)₂ After Coating the ELISA Plates with the T-2 in the Cell Supernatant

The hybridoma cells that can secret T-2 antibody were cultured using fresh medium (80% RPMI1640, 20% fetal bovine serum), and the cell culture supernatant was collected two days later.

Using CBS pH 9.6, the Fab′, F(ab)₂ and T-1 intact antibodies were diluted to 1 μg/mL, 1 μg/mL and 1.36 mg/mL, respectively, so that the molar concentration is uniform. Added them to each plate at 100 μL/well, 3 wells for each antibody, incubated overnight at 4° C., and washed three times with PBST (containing 0.05% Tween-20 in PBS buffer, pH 7.4). Added 200 μl of 1% OVA (ovalbumin) to incubate at 37° C. for 2 h to block the un-reacted site on the ELISA plate and washed once with PBST. 50 μL of 0.02 μg/mL, 0.2 μg/mL and 2 μg/mL TPO antigens and 50 μL of cell culture supernatant were added to each group, respectively, incubated at 37° C. for 1 h, and washed three times with PBST. Added 100 μL of 0.1 μg/mL HRP-labelled goat anti-mouse IgG Fc antibody, incubated at 37° C. for 1 h, and washed three times with PBST. Added 100 μL TMB substrate and incubated at room temperature for 10 min, and 50 μL of 2 M H₂SO₄ was added to terminate the reaction. The absorbance at a wavelength of 450 nm was measured.

Coating Effect of Fab′ and F(ab)₂

The anti-TPO antibody T-1 and Fab′ and F(ab)₂ thereof were used to coat the ELISA plates to examine their pairing with T-2 antibody. The sandwich ELISA was used to detect the TPO antigens with different concentrations. The results are shown in the below Table 1 (detection of the pairing of T-1 and Fab′ and F(ab)₂ thereof with T-2):

TABLE 1

| | 0.01 μg/mL | 0.1 μg/mL | 1 μg/mL |
|---|---|---|---|
| Fab′ | 1.96 | 2.65 | 2.74 |
| F(ab)₂ | 2.98 | 3.42 | 3.67 |
| T-1 | 3.09 | 3.64 | 3.78 |

Note:
The data in the table indicate OD₄₅₀, and the detection background value (under the same condition other than the absence of antigen) is less than 0.2.

The anti-TPO antibody T-1 and Fab′ and F(ab)₂ thereof were used to coat the ELISA plates. Used goat anti-mouse IgG Fc antibody as the second antibody in ELISA, and examined the pairing of three form of T-1 antibody with the T-2 antibody in the cell culture supernatant. The sandwich ELISA was used to detect the TPO antigens with different concentrations. The results are shown in the below Table 2 (detection of the pairing of T-1 and Fab′ and F(ab)₂ thereof with T-2 in the cell culture supernatant):

TABLE 2

| | 0.01 μg/mL | 0.1 μg/mL | 1 μg/mL |
|---|---|---|---|
| Fab′ | 1.46 | 2.33 | 2.65 |
| F(ab)₂ | 2.45 | 3.02 | 3.64 |
| T-1 | 3.85 | 3.79 | 3.83 |

Note:
The data in the table indicate OD₄₅₀, and the detection background value (under the same condition other than the absence of antigen) is less than 0.2.

### EXAMPLE 2

Preparation of Anti-Gastrin-17 Antibody Pair

Gastrin-17 (G17) is a polypeptide with 17-amino acid and is of great diagnostic significance for tumours and gastric diseases. In the art the double antibody sandwich method is recommended, but this molecule is too small, the preparation of an antibody pair has relatively high difficulty. In the previous studies, the inventors of the present application acquired a mouse-derived monoclonal antibody G-1 having an extremely high affinity with G17. In the present example, the paired antibodies of G-1 (IgG subtype) was prepared.

1) Acquisition of F(ab)₂

Reference is made to the method provided by Short Protocols in Immunology (John E. Coligan, USA, 2009) to use the pepsin digestion. Specifically, 2 mL of 3 mg/mL purified G-1 was dialyzed against 200 mL of acetic acid buffer (pH 4.0) at 4° C. for 4 h, the concentration was re-measured and adjusted to 2 mg/mL using acetate buffer with the same pH. The pepsin concentration of 0.1 mg/mL was formulated with the acetate buffer (pH 4.0), and 1 mL of the solution was added to the above described antibody solution to make the enzyme to antibody of 1:20 to react at 37° C. for 4 h. The reaction was terminated by adding 100 μL of 2 mol/L Tris base. The mixture was transferred to a dialysis bag and dialyzed against 1 L PBS of pH 8.0 at 4° C. The dialysate was loaded onto a 5 mm×100 mm protein A cross-linked agarose gel CL-4B chromatographic column and the unbound effluent is collected. The effluent was concentrated to 4 mL and loaded onto a 26 mm×900 mm polyacrylamide dextran S-200 Superfine chromatographic column to collect the component with the molecular weight of 110 kDa. The above described product was identified using 10% SDS non-reducing polyacrylamide gel electrophoresis, showing a band only at 110 kDa, a single band at 25 kDa was shown using non-reducing polyacrylamide gel electrophoresis. A280 was detected to determine the final concentration of F(ab)₂.

2) Screening the Antibodies that can be Paired with G-1 by the Sandwich ELISA Method

The mature Balb/c mice immunized with G17-KLH were challenged by intraperitoneal injection of 300 _{K}g of G17-KLH conjugate three days in advance. The spleen cells were harvested and ground to obtain lymphocyte, and the cell fusion of the lymphocyte and mice myeloma cell sp2/0 was facilitated by using PEG1500. Put them in 20 pieces of 96-well cell culture plates after fusion. Cultured at 37° C., 5% CO₂ for 7 days, replaced the fresh medium (80% RPMI1640, 20% fetal bovine serum) and cultured for another day.

The G-1 F(ab)₂ solution was diluted to 1μg/mL with a pH 9.6 carbonate buffer and was added to 20 pieces of 96-well ELISA plates at 100 μl/well, incubated at 4° C. for 12 h, washed with PBST (containing 0.05% Tween-20 in PBS buffer, pH 7.4). Added 200 μl of 1% OVA (ovalbumin) to incubate at 37° C. for 2 h to block the un-reacted site on the ELISA plate and washed once with PBST. 50 μL of 1 ng/mL G17 antigen and 50 μL of cell culture solution were added to each well, incubated at 37° C. for 1 h and washed three times with PBST. Added 100 μL of 0.1 μg/mL HRP-labelled goat anti-mouse IgG Fc antibody, incubated at 37° C. for 1 h, and washed three times with PBST. Added 100 μL TMB substrate and incubated at room temperature for 10 min. Added 50 μL of 2 M H₂SO₄ to terminate the reaction. The absorbance at a wavelength of 450 nm was measured and recorded as OD₁. Cell cultures with OD₁>1.5 were subjected to the detection of the next two steps.

3) Detecting the Relative Amounts of Total Antibodies in the Cell Cuture Supernatant by the Double Antibody Sandwich ELISA Method

The mixture of goat anti-mouse IgG1, IgG2a, IgG2b, IgG3 antibody (no cross-over from each other) was diluted to 2 μg/mL with a pH 9.6 carbonate buffer. Added them to ELISA plates at 100 μl/well, incubated at 4° C. for 12 h, washed with PBST (containing 0.05% Tween-20 in PBS buffer, pH 7.4). Added 200 μL of 1% OVA (ovalbumin), incubated at 37° C. for 2 h, and washed once with PBST. 100 μL of the cell culture solution (10-fold diluted with PBS) was added to each well, incubated at 37° C. for 1 h, and washed three times with PBST. Added 100 μL of 0.1 μg/mL HRP-labelled goat anti-mouse IgG antibody, incubated at 37° C. for 1 h, and washed three times with PBST. Added 100 μL TMB substrate and incubated at room temperature for 10 min. Added 50 μL of 2 M H₂SO₄ to terminate the reaction. The absorbance at a wavelength of 450 nm was measured and recorded as OD₂.

4) Detecting the Binding Strength of the Antibody in the Cell Culture Supernatant to the Antigen by the Indirect ELISA Method

The G17 antigen (a conjugate of G17 and BSA) was diluted to 1 μg/mL with a pH 9.6 carbonate buffer, added to ELISA plates at 100 μl/well, incubated at 4° C. for 12 h, washed three times with PBST. Added 200 μL of 1% OVA (ovalbumin), incubated at 37° C. for 2 h, and washed once with PBST. 100 μL of the cell culture solution (10-fold diluted with PBS) was added to each well, incubated at 37° C. for 1 h, and washed three times with PBST. Added 100 μL of 0.1 μg/mL HRP-labelled goat anti-mouse IgG antibody, incubated at 37° C. for 1 h, and washed three times with PBST. Added 100 μL TMB substrate and incubated at room temperature for 10 min, and 50 μL of 2 M H₂SO₄ was added to terminate the reaction. The absorbance at a wavelength of 450 nm was measured and recorded as OD₃.

5) Screening and Subcloning of Cells and the Large-Scale Preperation of the Target Antibody

The cultured cells were divided into two groups according to the single colony wells and the multiple colony wells. The first group was sorted according to OD₁/OD₂ from high to low, preferably the first ten strains of cells (named GS1-GS10 successively) were subject to an expansion culture.

The second group was sorted according to OD₁/OD₃ from high to low, and the first 20-well cells were subject to subcloning, respectively. The subcloned cells were detected according to the methods of 1) and 3), and ten cells with higher OD₁/OD₂ were selected (named GP1-GP10 successively) to be subject to an expansion culture.

A batch of Balb/c mice were injected intraperitoneally with 500 μL Freund's incomplete adjuvant 7 days in advance, and each mouse was injected with 0.5×10⁶ hybridoma cells of an expansion culture. Mice ascites was collected after seven days of feeding and the antibody was purified by n-caprylic acid-ammonium sulfate method.

6) The Application Effect of Antibody Pairs

Two detection systems were selected to evaluate the obtained antibody pairs.

ELISA platform: 1 μg/mL G-1 was used to coat ELISA plates, the newly selected antibody was labelled with HRP, and diluted with PBS to 0.1 μg/mL, and G17 antigen was detected by the sandwich method to evaluate the pairing effect.

Specifically, the previously screened antibody was labelled with HRP using the labelling method of the T2 antibody in Example 1. The antibody G-1 was diluted to 1 μg/mL with a pH 9.6 carbonate buffer. Added them to ELISA plates at 100 μl/well, incubated at 4° C. for 12 h, washed three times with PBST. Added 200 μL of 1% OVA, incubated at 37° C. for 2 h, and washed once with PBST. Added 50 μL of 0.1 ng/mL G17 antigen and 50 μL of 0.1 μg/mL HRP-labelled freshly prepared antibody to each well, incubated at 37° C. for 1 h and washed three times with PBST. Added 100 μL TMB substrate and incubated at room temperature for 10 min. Added 50 μL of 2 M H₂SO₄ to terminate the reaction. The absorbance at a wavelength of 450 nm was measured.

CLIA platform: G-1 was used to coat the magnetic micro-beads at a mass ratio of 1:100, and the newly screened antibody was labelled with ABEI, and different concentration of G17 was detected by the sandwich method to evaluate the pairing effect.

Specifically, 2 mg of the screened antibody was brought to 200 μL binding buffer (0.1 mol/L 2-[N-morpholino]ethanesulfonic acid, pH 4.5), 2 mg ABEI was dissolved into 500 μL binding buffer, and the two solutions were mixed; 10 mg EDC (diethylamine carbide) was weighted and added to the super-pure water, and 100 μL of the solution was immediately added to the ABEI antibody mixture in the last step, and incubated at room temperature for 2 h. The product was dialyzed against 4 L PBS at 4° C. for 8 h, up to the completion of the labelling. 1 mg of G-1 antibody and 100 mg of magnetic micro-beads were weighed in PBS, mixed and incubated at 40° C. for 2 h to complete the coating process. Added 20 μg magnetic micro-beads coated with G-1 antibody to the reaction cup of Maglumi 2000 full-automatic biochemical luminescence instrument, then added 100 μL of 0.1 ng/mL G17 antigen and 100 μL ABEI labelled antibody for reaction at 37° C. for 10 min. Cleaning solution was used to wash three times, 100 substrate A (NaOH) and substrate B (H₂O₂) were added and immediately sent to the measurement chamber to measure the relative luminous intensity Rlu.

The Pairing Results of 20 Antibodies Obtained in Example 2 with G-1

Through the preliminary screening of F(ab)₂ and goat anti-mouse IgG Fc, we obtained 371 wells of cells with OD₄₅₀>1.5, wherein 63 wells of cells were single colonies (single cell clumps formed by one cell growth division) and the remaining 308 wells of cells were multiple colonies. 10 strains of cells S1-S10 were selected from the 63 single colony wells through sorting according to OD₁/OD₂; 20 strains of cells were selected from the 278 multiple colony wells through sorting according to OD₁/OD₃, and further selected 10 strains of cells P1-P10 by sorting according to OD₁/OD₂ after subcloning.

G-1 was diluted to 1 μg/mL using CBS (pH 9.6), 100 μL of which was taken to coat the ELISA plates, GS1-GS10 and GP1-GP10 were labelled with HRP, added 100 μL at 0.1 μg/mL, and 100 μg/mL of G17 antigen was detected by the sandwich ELISA method. The results showed that all the antibodies were well paired with G-1, and all OD₄₅₀ were greater than 2.5 except for GS7, GS8, GS9 and GS10.

Table 3 shows the pairing of GS1-GS10, GP1-GP10 with G-1 evaluated by the sandwich ELISA method.

TABLE 3
The concentration of antigen

| | GS1 | GS2 | GS3 | GS4 | GS5 |
|---|---|---|---|---|---|
| 100 pg/mL | 3.29 | 3.31 | 3.12 | 2.71 | 2.76 |
| | GS6 | GS7 | GS8 | GS9 | GS10 |
| 100 pg/mL | 2.56 | 2.47 | 2.25 | 2.08 | 2.14 |
| | GP1 | GP2 | GP3 | GP4 | GP5 |
| 100 pg/mL | 3.43 | 3.52 | 3.38 | 3.34 | 3.32 |
| | GP6 | GP7 | GP8 | GP9 | GP10 |
| 100 pg/mL | 3.25 | 3.02 | 2.96 | 2.83 | 2.91 |

Note:
The data in the table indicate OD₄₅₀, and the detection background value (under the same condition other than the absence of antigen) is less than 0.2.

G-1 was used to coat the magnetic micro-beads produced by Snibe company, GS1-GS10 and GP1-GP10 were labelled with ABEI, and 100 μg/mL G17 antigen was detected by the sandwich method using the Snibe Maglumi system. The results show that GS2, GP1 and GP8 were paired well with G1, among which GS2 and GP1 had the best effect.

Table 4 shows the pairing of GS1-GS10, GP1-GP10 with G-1 evaluated by the sandwich CLIA method.

TABLE 4
The concentration of antigen

| | GS1 | GS2 | GS3 | GS4 | GS5 |
|---|---|---|---|---|---|
| 0 pg/mL | 809 | 2430 | 5240 | 30907 | 18973 |
| 100 pg/mL | 43802 | 1107631 | 23464 | 58573 | 19645 |
| | GS6 | GS7 | GS8 | GS9 | GS10 |
| 0 pg/mL | 3972 | 13451 | 3451 | 8512 | 5634 |
| 100 pg/mL | 8716 | 22142 | 5632 | 14421 | 6421 |
| | GP1 | GP2 | GP3 | GP4 | GP5 |
| 0 pg/mL | 2871 | 14512 | 16752 | 12133 | 13763 |
| 100 pg/mL | 1386542 | 56214 | 47532 | 245231 | 21765 |
| | GP6 | GP7 | GP8 | GP9 | GP10 |
| 0 pg/mL | 8965 | 5674 | 3747 | 16785 | 1652 |
| 100 pg/mL | 13341 | 8532 | 464673 | 18551 | 2043 |

Note:
The data in Table 4 indicate the relative luminescent intensity.

### EXAMPLE 3

The Preparation of Anti-Folate Binding Protein Antibody Pair

Folic acid binding protein (FABP), also known as folic acid receptor protein, plays an important role in cell division, proliferation and sound field, and is generally detected by using the double antibody sandwich method. In the previous work we obtained an antibody F-1 with a relatively high affinity for FABP. In this example we would prepare the pairing antibody of F-1.

1) Acquisition of F(ab)₂

2 mL of 3 mg/mL purified F-1 was dialyzed against 200 mL of acetic acid buffer (pH 4.0) at 4° C. for 4 h, the concentration was re-measured and adjusted to 2 mg/mL using acetate buffer with the same pH. The pepsin concentration of 0.1 mg/mL was formulated with the acetate buffer (pH 4.0), and 1 mL of the solution was added to the above described antibody solution to make the enzyme to antibody of 1:20 to react at 37° C. for 4 h. The reaction was terminated by adding 100 μL of 2 mol/L Tris base. The mixture was transferred to a dialysis bag and dialyzed against 1 L PBS of pH 8.0 at 4° C. The dialysate was loaded onto a 5 mm×100 mm protein A cross-linked agarose gel CL-4B chromatographic column and the unbound effluent is collected. The effluent was concentrated to 4 mL and loaded onto a 26 mm×900 mm polyacrylamide dextran S-200 Superfine chromatographic column to collect the component with the molecular weight of 110 kDa. The above described product was identified using 10% SDS non-reducing polyacrylamide gel electrophoresis, showing a band only at 110 kDa, a single band at 25 kDa was shown using non-reducing polyacrylamide gel electrophoresis. A280 was detected to determine the final concentration of F(ab)₂.

2) Screening the Antibodies that can be Paired with F-1 by the Sandwich ELISA Method

The mature Balb/c mice immunized with FABP were challenged by intraperitoneal injection of 300 μg of FABP three days in advance. The spleen cells were harvested and ground to obtain lymphocyte, and the cell fusion of the lymphocyte and mice myeloma cell sp2/0 was facilitated by using PEG1500. Put them in 20 pieces of 96-well cell culture plates after fusion. Cultured at 37° C., 5% CO₂ for 7 days, replaced the fresh medium (80% RPMI1640, 20% fetal bovine serum) and cultured for another day.

The F-1 F(ab)₂ solution was diluted to 1 μg/mL with a pH 9.6 carbonate buffer and was added to 20 pieces of 96-well ELISA plates at 100 μl/well, incubated at 4° C. for 12 h, and washed with PBST (containing 0.05% Tween-20 in PBS buffer, pH 7.4). Added 200 μL of 1% OVA (ovalbumin) to incubate at 37° C. for 2 h to block the un-reacted site on the ELISA plate and washed once with PBST. 50 μL of 50 ng/mL FABP antigen and 50 μL of cell culture solution were added to each well, incubated at 37° C. for 1 h and washed three times with PBST. Added 100 μL of 0.1 μg/mL HRP-labelled goat anti-mouse IgG Fc antibody, incubated at 37° C. for 1 h, and washed three times with PBST. Added 100 μL TMB substrate and incubated at room temperature for 10 min. Added 50 μL of 2 M H₂SO₄ to terminate the reaction. The absorbance at a wavelength of 450 nm was measured and recorded as OD₁. Cell cultures with OD₁>1.5 were subjected to the detection of the next two steps.

3) Detecting the Relative Amount of Total Antibodies in the Cell Culture Supernatant by the Double Antibody Sandwich ELISA Method

The mixture of goat anti-mouse IgG1, IgG2a, IgG2b, IgG3 antibodies (no cross-link from each other) was diluted to 2 μg/mL with a pH 9.6 carbonate buffer. Added them to ELISA plates at 100 μl/well, incubated at 4° C. for 12 h, and washed with PBST (containing 0.05% Tween-20 in PBS buffer, pH 7.4). Added 200 μL of 1% OVA (ovalbumin), incubated at 37° C. for 2 h, and washed once with PBST. 100 μL of the cell culture solution (10-fold diluted with PBS) was added to each well, incubated at 37° C. for 1 h, and washed three times with PBST. Added 100 μL of 0.1 μg/mL HRP-labelled goat anti-mouse IgG antibody, incubated at 37° C. for 1 h, and washed three times with PBST. Added 100 μL TMB substrate and incubated at room temperature for 10 min, and 50 μL of 2 M H₂SO₄ was added to terminate the reaction. The absorbance at a wavelength of 450 nm was measured and recorded as OD₂.

4) Detecting the Binding Strength of the Antibody in the Cell Culture Supernatant to the Antigen by the Indirect ELISA Method

The FABP antigen was diluted to 1 μg/mL with a pH 9.6 carbonate buffer. Added them to ELISA plates at 100 μl/well, incubated at 4° C. for 12 h, and washed three times with PBST. Added 200 μL of 1% OVA (ovalbumin), incubated at 37° C. for 2 h, and washed once with PBST. 100 μL of the cell culture solution (10-fold diluted with PBS) was added to each well, incubated at 37° C. for 1 h, and washed three times with PBST. Added 100 μL of 0.1 μg/mL HRP-labelled goat anti-mouse IgG antibody, incubated at 37° C. for 1 h, and washed three times with PBST. Added 100 μL TMB substrate and incubated at room temperature for 10 min. Added 50 μL of 2 M H₂SO₄ to terminate the reaction. The absorbance at a wavelength of 450 nm was measured and recorded as OD₃.

5) Screening and Subcloning of Cells and the Large-Scale Preperation of the Target Antibody

The cultured cells were divided into two groups according to the single colony wells and the multiple colony wells. The first group was sorted according to OD₁/OD₂ from high to low, preferably the first ten cell strains (named FS1-FS10 successively) were subject to an expansion culture.

The second group was sorted according to OD₁/OD₃ from high to low, and the first 20-well cells were subject to subcloning, respectively. The subcloned cells were detected according to the methods of 1) and 3), and ten cells with higher OD₁/OD₂ were are selected (named FP1-FP10 successively) to be subject to an expansion culture.

A batch of Balb/c mice were injected intraperitoneally with 500 μL Freund's incomplete adjuvant 7 days in advance, and each mouse was injected with 0.5×10⁶ hybridoma cells of expansion culture. Mice ascites was collected after seven days of feeding and the antibody was purified by n-caprylic acid-ammonium sulfate method.

6) The Application Effect of Antibody Pairs

We selected two detection systems to evaluate the obtained antibody pairs.

ELISA platform: 1 μg/mL F-1 was used to coat ELISA plates, the newly selected antibody was labelled with HRP, and diluted with PBS to 0.1 μg/mL, and FABP antigen was detected by the sandwich method to evaluate the pairing effect.

Specifically, the previously screened antibody was labelled with HRP using the labelling method of the T2 antibody in Example 1. The antibody F-1 was diluted to 1 μg/mL with a pH 9.6 carbonate buffer. Added them to ELISA plates at 100 μl/well, incubated at 4° C. for 12 h, and washed three times with PBST. Added 200 μL of 1% OVA, incubated at 37° C. for 2 h, and washed once with PBST. Added 50 μL of 50 ng/ml FABP and 50 μL of 0.1 μg/mL HRP-labelled freshly prepared antibody to each well, incubated at 37° C. for 1 h and washed three times with PBST. Added 100 μL TMB substrate and incubated at room temperature for 10 min. Added 50 μL of 2 M H₂SO₄ to terminate the reaction. The absorbance at a wavelength of 450 nm was measured.

CLIA platform: F-1 was used to coat the magnetic micro-beads at a mass ratio of 1:100, and the newly FS1-FS10 and FP1-FP10 antibody was labelled with ABEI, and FABP antigen was detected by the sandwich method to evaluate the pairing effect.

Specifically, 2 mg of the screened antibody was brought to 200 μL binding buffer (0.1 mol/L 2-[N-morpholino]ethanesulfonic acid, pH 4.5), 2 mg ABEI was dissolved into 500 μL binding buffer, and the two solutions were mixed; 10 mg EDC (diethylamine carbide) was weighted and added to the super-pure water, and 100 μL of the solution was immediately added to the ABEI antibody mixture obtained in the last step, and incubated at room temperature for 2 h. The product was dialyzed against 4 L PBS at 4° C. for 8 h, up to the completion of the labelling. 1 mg of N-1 antibody and 100 mg of magnetic micro-beads produced by Snibe company were weighed and added to PBS to be mixed, and incubated at 40° C. for 2 h to complete the coating process. The magnetic micro-beads coated with N-1 antibody were loaded to the reaction cup using Maglumi full-automatic biochemical luminescence instrument, and then added 100 μL of 50 ng/ml FABP and 100 μL ABEI labelled antibody for reaction at 37° C. for 10 min. Cleaning solution was used to wash three times, 100 substrate A (NaOH) and substrate B (H₂O₂) were added and immediately sent to the measurement chamber to measure the relative luminous intensity Rlu.

The Pairing Results of the 20 Antibodies Obtained in Example 3 with F-1

Through the preliminary screening of F(ab)₂ and goat anti-mouse IgG Fc, we obtained 480 wells of cells with OD₄₅₀>1.5, wherein 72 wells of cells were single colonies (single cell clumps formed by one cell growth division) and the remaining 408 wells of cells were multiple colonies. 10 cell strains FS1-FS10 were selected from the 72 single colony wells through sorting according to OD₁/OD₂; 20 strains of cells were selected from 408 multiple colony wells through sorting according to OD₁/OD₃, and 10 strains of cells FP1-FP10 were further selected by sorting according to OD₁/OD₂ after subcloning.

F-1 was diluted to 1 μg/mL using CBS (pH 9.6), 100 μL of which was taken to coat the ELISA plates, FS1-FS10 and FP1-FP10 were labelled with HRP and diluted to 0.1 μg/mL using PBS, 100 μL of which was added to each well, and 50 ng/mL FABP was detected by the sandwich ELISA method. The results showed that all the antibodies were well paired with F-1, and all OD₄₅₀ were greater than 2.5 except for GS7, GS8, GS9 and GS10.

Table 5 shows the pairing of FS1-FS10 and FP1-FP10 with F-1 evaluated by the sandwich ELISA method.

TABLE 5
The concentration of antigen

| | FS1 | FS2 | FS3 | FS4 | FS5 |
|---|---|---|---|---|---|
| 50 ng/ml | 3.82 | 3.56 | 3.11 | 2.65 | 2.78 |
| | FS6 | FS7 | FS8 | FS9 | FS10 |
| 50 ng/ml | 2.68 | 2.52 | 2.32 | 2.41 | 2.14 |
| | FP1 | FP2 | FP3 | FP4 | FP5 |
| 50 ng/ml | 3.75 | 3.82 | 3.85 | 3.46 | 3.65 |
| | FP6 | FP7 | FP8 | FP9 | FP10 |
| 50 ng/ml | 3.55 | 3.46 | 2.98 | 3.07 | 3.11 |

Note:
The data in the table indicate OD₄₅₀, and the detection background value (under the same condition other than the absence of antigen) is less than 0.2.

F-1 was used to coat the magnetic micro-beads produced by Snibe company, FS1-FS10 and FP1-FP10 were labelled with ABEI, and 50 ng/ml FABP antigen was detected by the sandwich method in the Maglumi system. The results showed that FS-3, FP-1, FP-2, FP-5, and FP-7 were paired well with F-1, among which FS-3, FP-1 and FP-7 had the best effect.

Table 6 shows the pairing of FS1-FS10 and FP1-FP10 with F-1 evaluated on the CLIA platform.

TABLE 6
The concentration of antigen

| | FS1 | FS2 | FS3 | FS4 | FS5 |
|---|---|---|---|---|---|
| 0 pg/ml | 5896 | 12474 | 8672 | 35278 | 6788 |
| 50 ng/ml | 173110 | 214054 | 1836981 | 82578 | 124274 |
| | FS6 | FS7 | FS8 | FS9 | FS10 |
| 0 pg/ml | 8677 | 12571 | 8674 | 6587 | 5874 |
| 50 ng/ml | 65814 | 24157 | 10254 | 6438 | 9375 |
| | FP1 | FP2 | FP3 | FP4 | FPS |
| 0 pg/ml | 5387 | 28797 | 16975 | 6837 | 7854 |
| 50 ng/ml | 1987752 | 988725 | 268774 | 367784 | 1355789 |
| | FP6 | FP7 | FP8 | FP9 | FP10 |
| 0 pg/ml | 24875 | 8074 | 8982 | 12745 | 4387 |
| 50 ng/ml | 687724 | 1386489 | 35047 | 254774 | 82449 |

Note:
The data in Table 4 indicate the relative luminescent intensity.

### COMPARATIVE EXAMPLE 4

FABP Antibody Pairs were Prepared Using the Commonly Used Method

The commonly used strategies are as the method of preparing antibody pairs described in the reference “The preparation of anti-human alpha-fetoprotein monoclonal antibody and the establishment of double antibody sandwich ELISA detection technology, Sun Yifan, Chinese Medicinal Biotechnology, August 2014, Vol. 9, No. 4”.

1) Acquisition of Hybridoma Cells

The mature Balb/c mice immunized with FABP were challenged by intraperitoneal injection of 300 μg of FABP three days in advance. The spleen cells were harvested and ground to obtain lymphocyte, and the cell fusion of the lymphocyte and mice myeloma cell sp2/0 was facilitated by using PEG1500. Put them in 10 pieces of 96-well cell culture plates after fusion. Cultured at 37° C., 5% CO₂ for 7 days, replaced the fresh medium (80% RPMI1640, 20% fetal bovine serum) and cultured for another day.

2) Screening the Antibodies that Bind to FABP Antigens

The FABP was diluted to 1 μg/mL using a pH 9.6 carbonate buffer and was added to the ELISA plates at 100 μl/well, incubated at 4° C. for 12 h, and washed three times with PBST (containing 0.05% Tween-20 in PBS buffer, pH 7.4). Added 200 μL of 1% OVA (ovalbumin), incubated at 37° C. for 2 h, and washed once with PBST. 100 μL of the above described cell culture solution (10-fold diluted with PBS) was added to each well, incubated at 37° C. for 1 h, and washed three times with PBST. Added 100 μL of 0.1 μg/mL HRP-labelled goat anti-mouse IgG antibody, incubated at 37° C. for 1 h, and washed three times with PBST. Added 100 μL TMB substrate and incubated at room temperature for 10 min. Added 50 μL of 2 M H₂SO₄ to terminate the reaction. The absorbance at a wavelength of 450 nm was measured. 20-well cells were selected according to the absorbance from high to low.

3) Screening Paired Antibodies

Twenty strains of cells were subcloned, and the individual cell strains were isolated and subject to an expansion culture. A large amount of antibodies secreted by each cell strain were obtained as that in Example 1, and the 20 antibodies obtained were named FE1-FE20 successively.

A total of 21 antibodies of FE1-FE20 and N1 were combined in pairs (also can be self-combined), forming 441 (21×21) combinations. In an antibody pair of these combinations, one was used to coat the ELISA plate and another one was labelled with HRP. The FABP antigens were detected by the sandwich ELISA method. The cells with positive results were detected (OD450>negative control wells±double variance, negative control wells are all the same except of absence of antigen). The procedure of labelling and detection is the same as that in Example 3.

4) The Application Effect of Antibody Pairs

Using the CLIA sandwich method, the pairing successful antibody pairs were tested for further pairing against the Maglumi system of Snibe company. Specifically, using the method in Example 3, one of an antibody pair was used to coat the magnetic micro-beads produced by Snibe and another one was labelled with ABEI. The FABP was detected using the Maglumi 2000 sandwich method to make evaluation.

The Pairing Results of the 20 Antibodies Obtained in the Comparative Example

1321 wells of cells were screened by the indirect method and detected to be positive (OD₄₅₀>1.5), and among which 20 strains with relatively high binding value were selected for paired detection. A total of 21 antibodies of FE1-FE20 and F-1 were pair-matched into 441 groups. One antibody of each group was labelled with HRP and another one was used to coat the ELISA plate. 50 ng/ml FABP was detected by the sandwich ELISA method. The pairing results showed that there were 62 combinations with OD₄₅₀>1.5, of which only 6 combinations had OD₄₅₀>2.5: FE2/FE12, FE2/FE17, FE5/FE13, FE7/FE14, FE8/FE18 and FE10/F-1.

Table 7 shows the screening of anti-FABP antibody pairs using the conventional method.

TABLE 7

| | FE12 | FE13 | FE14 | FE17 | FE18 | F-1 |
|---|---|---|---|---|---|---|
| FE2 | 2.84 | 0.21 | 0.13 | 3.01 | 0.74 | 0.67 |
| FE5 | 0.24 | 2.69 | 0.29 | 0.18 | 0.67 | 1.67 |
| FE7 | 0.28 | 0.67 | 2.98 | 0.21 | 0.32 | 0.43 |
| FE8 | 0.11 | 0.18 | 0.26 | 0.19 | 2.56 | 0.58 |
| FE10 | 0.28 | 0.07 | 0.18 | 0.24 | 0.47 | 3.12 |

Each of the above six antibody pairs were labelled with ABEI and used to coat magnetic beads, respectively, and 50 ng/mL FABP antigen was detected by the sandwich method on Maglumi 2000. The results showed that only FE2/FE17 was paired to some extent, and the other antibody pairs were not suitable for Maglumi system.

Table 8 shows the evaluation of the pairing of six antibody pairs by the CLIA sandwich method.

TABLE 8

| | | FE2/ | | | FE8/ | FE10/ |
|---|---|---|---|---|---|---|
| | FE2/FE12 | FE17 | FE5/FE13 | FE7/FE14 | FE18 | F-1 |
| 0 pg/ml | 12841 | 8941 | 6874 | 5681 | 6624 | 12571 |
| 50 ng/ml | 26574 | 541001 | 12574 | 9824 | 8974 | 35714 |

Result Analysis

As can be seen from Table 1 in Example 1, antibody T-1 could continue to be paired with T-2 for sandwich detection of TPO antigen after it was digested into Fab′ fragment and F(ab)₂ by enzyme, wherein the pairing effect of F(ab)₂ was close to that of the intact antibody. In Table 2, the T-2 antibody was present in the cell culture supernatant. The HRP-labelled goat anti-mouse IgG Fc was used as the second antibody. If the intact T-1 was used as the capture antibody, since T-1 and T-2 have the same Fc fragment, the second antibody reacted with both of them. The detection value did not reduce with the decrease of the addition of the antigen, and thus cannot reflect the true pairing. However, if the Fab′ fragment and F(ab)₂ were used as the capture antibody, the detection values were relatively high and reduced with the decrease of the addition of the antigen, indicating that both the Fab′ fragment and F(ab)₂ of T-1 were paired well with the T-2 antibody in the cell culture supernatant and could be well detected. As the stability of F(ab)₂ was better than that of the monovalent Fab′, it reflects the better pairing effect. To sum up, using the Fab′ fragment and F(ab)₂ of an antibody as the capture antibody, it is possible to directly screen for antibody from the cell culture supernatant with which it is paired.

From Examples 2 and 3, it was found that all of the screened antibody could be used in the sandwich ELISA method, and the pairing strength was roughly in accordance with the sorting order during the screening, indicating that the optimized strategy was reasonable. From Tables 2 and 4, it was found that the resulting antibodies after two rounds of screening (GP1-GP10 and FP1-FP101) were superior to those of single-round screening (GS1-GS10 and FS1-FS10), indicating that the pairing success rate was improved to some extent through two-round-screening of OD₁/OD₃ and OD₁/OD₂ compared with that of OD₁/OD₂ single-round-screening.

Screening was performed using all the cell colonies (5,000-10,000) of one cell fusion by the sandwich method in Example 3 and 408 wells of cells were obtained. All the 20 strains of cells through screening can be paired with the existing high-sensitivity antibody F-1 to be used in the sandwich ELISA method, among which five strains can be paired with F-1 to be used in the sandwich CLIA method. The comparative Example firstly screened 20 antibodies that can bind to the antigen, and then these antibodies were prepared in large-scale, purified and labelled with HRP. Only six antibody pairs that can be used in the sandwich ELISA method were screened from the 441 combinations by the chessboard method, among which one antibody pair can be used in the CLIA sandwich method.

The method that antibody coats the ELISA plate in ELISA is the physical adsorption, the used marker HRP is macromolecular protein, and generally takes a 1:1 molar ratio when it labels antibody; however, the process that antibody coats magnetic beads in CLIA and ratio that the antibody is labelled with small molecule compounds ABEI are all of relatively large difference from that in ELISA, and so the impact on the nature of the antibody itself is not the same. In addition, CLIA amplification signal function far exceeds the ELISA, and the requirements on the sensitivity of the antibody are higher. Therefore, only a small fraction of the antibody pairs that can be applied to ELISA are suitable for CLIA.

Comparing Example 3 and the comparative Example, we found that the former only required the use of antibody in the cell culture supernatant when performing the pairing screening, while the latter required the prepared antibodies; the former did not need the purification and labelling of antibody in the screening process, while the latter must use the antibodies purified and labelled with HRP; similarly for the 20 obtained antibodies, in the former all could be paired with the existing high-sensitivity antibodies, while the latter obtained only six antibody pairs, of which only one could be paired with the existing high-sensitivity antibodies; the former obtained five antibody pairs that can be used in the CLIA sandwich method, while the latter obtained only one pair with the pairing effect not as good as the former.

From the above description, it can be seen that the above-described examples of the present application achieved the following technical effects:

1) The pairing detection can be directly performed with the cell culture supernatant, which greatly expands the source of antibody to be detected and does not need labelling and purification in the screening process;

2) When too many antibodies are obtained in the preliminary screening, effective evaluation system is established for further screening, which saves manpower while greatly increasing the success rate.

The above-mentioned are merely several preferred embodiments of the present application, which are not intended to limit the present application, and it will be apparent to those skilled in the art that various modifications and variations can be made in the present application. Any modifications, equivalent replacements, improvements and the like made within the spirit and principle of the present application shall fall within the scope of the present application.

A method for preparing an antibody pair, comprising: using an antigen-binding fragment of an existing antibody as a capture antibody and using an anti-crystallizable fragment antibody as a labelled antibody to directly screen a target antibody which can be paired with said existing antibody from cell culture supernatant.

2. The method according to claim 1, wherein the antigen-binding fragment of said existing antibody is Fab′ or F(ab′)₂, preferably F(ab′)₂; and said labelled antibody is an anti-Fc antibody.

3. The method according to claim 1, further comprising: optimizing the screened antibodies according to an total antibody content in said cell culture supernatant or an binding strength of an antibody to be screened in said cell culture supernatant to a target antigen.

4. The method according to claim 3, wherein said labelled antibody is labelled with a tracer marker, which directly or indirectly labels said labelled antibody;
preferably, said tracer marker is at least one selected from the group consisting of an enzyme label, a fluorescent dye, a chemiluminescent dye, and a radioactive label.

5. The method according to claim 4, wherein said optimizing step comprises:
a) detecting the screened antibodies that can be paired with the capture antibody in the cell culture supernatant by the sandwhich method, and recording the resulting signal as OD₁;
b) detecting the total antibody content in said cell culture supernatant, and recording the resulting signal as OD₂;
c) detecting the binding strength of the antibody to be screened in said cell culture supernatant to the target antigen, and recording the resulting signal as OD₃; and
d) evaluating and optimizing the antibody screened in said step a) based on an ratio of OD₁/OD₂ or OD₁/OD₃.

6. The method according to claim 5, wherein said step d) comprises:
1) if the cell culture is a single cell strain cell culture, then the antibody with higher OD₁/OD₂ is paired better with said existing antibody;
2) if the cell culture is a multiple cell strains cell culture, then the cell culture with higher OD₁/OD₃ is selected to be separated into single cells for continuous culture so as to obtain new cell cultures, and the new cell cultures are further optimized following said step 1).

7. The method according to claim 6, wherein said step d) specifically comprises:
dividing said cell cultures into two groups according to single colony wells and multiple colony wells;
sorting the group of the single colony wells according to OD₁/OD₂ from high to low, and selecting the first N cell strains, wherein said N is 1-20;
sorting the group of the multiple colony wells according to OD₁/OD₃ from high to low, and selecting the first M cell wells for subcloning, respectively, and then detecting the subcloned cells according to the methods of said step a) and said step b) and selecting N cell strains with higher OD₁/OD₂, wherein said M is 1-40.

8. The method according to claim 6, wherein said step b) specifically comprises:
detecting the total antibody content in the cell culture supernatant using an antibody that specifically binds to an antibody conserved region or an immunoglobulin binding protein;
preferably, said antibody that specifically binds to an antibody conserved region is a mixture of two or more strains selected from the group consisting of goat anti-mouse IgM, goat anti-mouse IgA, goat anti-mouse IgD, goat anti-mouse IgG1, goat anti-mouse IgG2a, goat anti-mouse IgG2b, goat anti-mouse IgG3, rabbit anti-mouse IgM, rabbit anti-mouse IgA, rabbit anti-mouse IgD, rabbit anti-mouse IgG1, rabbit anti-mouse IgG2a, rabbit anti-mouse IgG2b and rabbit anti-mouse IgG3, and said immunoglobulin binding protein is a Staphylococcus protein A and/or a Streptococcus protein G

9. The method according to claim 6, wherein said step c) specifically comprises:
coating the target antigen on a solid phase, and detecting the binding strength of the antibody to be screened in said cell culture supernatant to the target antigen by means of a second antibody.

10. The method according to claim 6, wherein said existing antibody is any one selected from the group consisting of an anti-gastrin-17 antibody, an anti-folate binding protein antibody, an anti-thyroid peroxidase antibody, an anti-thyroglobulin antibody, an anti-insulin antibody, an anti-ferritin antibody, an anti-alpha-fetoprotein antibody, an anti-carcinoembryonic antibody, an anti-prostate specific antigen antibody, an anti-luteinizing hormone antibody, an anti-prolactin antibody, an anti-human chorionic gonadotropin antibody, an anti-neuron-specific enolase antibody, an anti-carbohydrate antigen 125 antibody, an anti-carbohydrate antigen 153 antibody, an anti-carbohydrate antigen 199 antibody, an anti-cytokeratin nineteen fragment antibody, an anti-carbohydrate antigen 724 antibody, an anti-carbohydrate antigen 242 antibody, an anti-growth hormone antibody, an anti-myoglobin antibody, an anti-carbohydrate antigen 50 antibody, an anti-C reactive Protein antibody, an anti-corticotropin antibody, an anti-creatine kinase isoenzyme antibody, an anti-Sangtec-100 protein antibody, an anti-laminin antibody, an anti-type IV collagen antibody, an anti-brain natural peptide N-terminal precursor protein antibody, an anti-troponin antibody, an anti-parathyroid hormone antibody, an anti-calcitonin antibody, an anti-procalcitonin antibody, an anti-prostate acid phosphatase antibody, an anti-osteocalcin antibody, an anti-pregnancy-associated protein A antibody, an anti-pepsinogen I antibody, an anti-pepsinogen II antibody, an anti-insulin-like growth factor antibody, or an anti-D-dimer antibody.

11. The method according to claim 1, further comprising expansion culture of the cell culture containing the screened antibody and large-scale preparation and purification of the target antibody.

12. A kit for preparing an antibody pair, comprising:
a capture antibody, which is formed by immobilizing the antigen-binding fragment of an existing antibody on a solid phase medium; and
a labelled antibody, which is an anti-crystallizable fragment antibody.

13. The kit according to claim 12, wherein the antigen-binding fragment of said existing antibody is Fab′ or F(ab′)₂, preferably F(ab′)₂; and said labelled antibody is an anti-Fc antibody.

14. The kit according to claim 12, wherein said labelled antibody is labelled with a tracer marker, which directly or indirectly labels said labelled antibody;
preferably, said tracer marker is at least one selected from the group consisting of an enzyme label, a fluorescent dye, a chemiluminescent dye, and a radioactive label.

15. The kit according to claim 12, wherein further comprising: an antibody that specifically binds to an antibody conserved region or an immunoglobulin binding protein;
preferably, said antibody that specifically binds to an antibody conserved region is a mixture of two or more strains selected from the group consisting of goat anti-mouse IgM, goat anti-mouse IgA, goat anti-mouse IgD, goat anti-mouse IgG1, goat anti-mouse IgG2a, goat anti-mouse IgG2b, goat anti-mouse IgG3, rabbit anti-mouse IgM, rabbit anti-mouse IgA, rabbit anti-mouse IgD, rabbit anti-mouse IgG 1, rabbit anti-mouse IgG2a, rabbit anti-mouse IgG2b, and rabbit anti-mouse IgG3, and said immunoglobulin binding protein is a Staphylococcus protein A and/or a Streptococcus G protein.

16. The kit according to claim 12, further comprising: a target antigen which directly or indirectly binds to a solid phase medium.

17. The kit according to claim 12, wherein said solid phase is an ELISA plate, magnetic beads, or colloidal gold.

18. The kit according to claim 12, wherein said existing antibody is any one selected from the group consisting of an anti-gastrin-17 antibody, an antifolate binding protein antibody, an anti-thyroid peroxidase antibody, an anti-thyroglobulin antibody, an anti-insulin antibody, an anti-ferritin antibody, an anti-alpha-fetoprotein antibody, an anti-carcinoembryonic antibody, an anti-prostate specific antigen antibody, an anti-luteinizing hormone antibody, an anti-prolactin antibody, an anti-human chorionic gonadotrnpin antibody, an anti-neuron-specific enolase antibody, an anti-carbohydrate antigen 125 antibody, an anti-carbohydrate antigen 153 antibody, an anti-carbohydrate antigen 199 antibody, an anti-cytokeratin nineteen fragment antibody, an anti-carbohydrate antigen 724 antibody, an anti-carbohydrate antigen 242 antibody, an anti-growth hormone antibody, an anti-myoglobin antibody, an anti-carbohydrate antigen 50 antibody, an anti-C reactive Protein antibody, an anti-corticotropin antibody, an anti-creatine kinase isoenzyme antibody, an anti-Sangtec-100 protein antibody, an anti-laminin antibody, an anti-type IV collagen antibody, an anti-brain natural peptide N-terminal precursor protein antibody, an anti-troponin antibody, an anti-parathyroid hormone antibody, an anti-calcitonin antibody, an anti-procalcitonin antibody, an anti-prostate acid phosphatase antibody, an anti-osteocalcin antibody, an anti-pregnancy-associated protein A antibody, an anti-pepsinogen I antibody, an anti-pepsinogen II antibody, an anti-insulin-like growth factor antibody or an anti-D-dimer antibody.

19. The kit according to claim 12, further comprising: reagents used in expansion culture of cell cultures and large-scale preparation and purification of the target antibody.

20. (canceled)

21. (canceled)

22. A system for preparing an antibody pair, comprising a kit according to claim 12 and a semi-automatic or full-automatic immunoassay analyzer.
